(19) United States
(12) Patent Application Publication
(10) Pub. No.: US 2008/0014230 A1
(43) Pub. Date: Jan. 17, 2008

(54) Cosmetic compositions combining a C-glycoside derivative and an N-acylamino amide derivative

(75) Inventors: Nathalie Pineau, Herblay (FR); Philippe Catroux, Guitres (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 11/822,098

(22) Filed: Jul. 2, 2007

Related U.S. Application Data
(60) Provisional application No. 60/832,942, filed on Jul. 25, 2006.

(30) Foreign Application Priority Data
Jul. 3, 2006 (FR) 06 06014

(51) Int. Cl. A61K 8/72; A61K 31/70; A61P 17/00; A61Q 17/00; A61Q 19/00

(52) U.S. Cl. 424/401; 424/59; 424/63; 424/64; 424/70.1; 514/25; 514/54

(57) ABSTRACT

The present invention relates to a cosmetic and/or dermatological composition comprising, in a physiologically acceptable medium, at least one N-acylamino amide derivative and at least one C-glycoside derivative.

This non provisional application claims the benefit of French Application No. 06 06014 filed on Jul. 3, 2006 and U.S. Provisional Application No. 60/832,942 filed on Jul. 25, 2006.

The present invention relates to the field of cosmetic and/or dermatological compositions.

More particularly, the invention is directed towards proposing novel cosmetic and/or dermatological compositions comprising a combination between an elastase-inhibiting compound of the N-acylamino amide family and at least one activator of glycosaminoglycan and of collagen VII synthesis, chosen from C-glycoside derivatives.

These compositions prove to be particularly advantageous for preventing and/or treating the signs of ageing and/or photo-ageing of the skin and in particular for increasing the epidermal thickness by increasing keratinocyte differentiation.

The epidermis is a keratinized stratified pavimentous epithelium. Its mean thickness ranges from 60 to 100 μm and may reach 600 to 700 μm on the sole of the feet and the palm of the hands. It consists mainly of keratinocytes (90%), but also other cells, and rests on a basal membrane that separates it from the dermis.

Progressive differentiation of cells of the basal membrane towards the surface of the epidermis is accompanied by a multitude of changes. By way of example, mention should be made of keratinization, i.e. the expression, synthesis and organization of various keratins, their interactions with other proteins and their changes throughout differentiation.

Differentiation, which is a genetically programmed process, is observed by microscopy by structural changes observed within various epidermal layers, which, starting from the basal membrane, are known as: basal, spiny, granular and horny. On differentiating, the cell becomes increasingly specialized and limited in its structures and functions.
Thus, the keratinocytes of the basal layer, connected to the dermo-epidermal junction via hemidesmosomes, are cubic or prismatic and are united together by desmosomes.
The desmosomes, which are abundant in the spiny layer, give the keratinocytes a spiny appearance. They become flattened and increase in volume in the spiny layer and also in the granular layer. The appearance of keratohyalin granules gives this layer its name, the keratinocytes of which also have lamellar bodies, organelles still undergoing active metabolism and desmosomes.
During the transition from the granular layer to the horny layer, the organelles disappear, the keratohyalin granules extend into large masses in the cytoplasm, the lamellar bodies overspill into the enlarged intercellular space, the keratin macrofibrils accumulate and the cytoplasmic membrane thickens.
Corneocytes are inert cell residues endowed with a robust keratin cytoskeleton and a protective horny sheath under the cytoplasmic membrane wrapped in intercellular lipids.
At the end of differentiation, the cell has become a lump of virtually inert keratins.

This keratinization property of keratinocytes, combined with the property of being highly connectable cells by virtue of their numerous cellular junctions, give the epidermis increased resistance to environmental attack. The cells of the horny layer, which are indeed dead but protective, desquamate and are replaced by others by virtue of the proliferation of the basal cells such that the cell loss and the cell multiplication are quite balanced.

During the menopause, the skin undergoes changes in all its compartments, i.e. dermal and epidermal.

The main changes concern the dermis and are a decrease in the collagen content and in the thickness of the dermis. In menopausal women, this results in thinning of the skin and/or of the mucous membranes. Women then experience a sensation of “dry skin” or of taut skin and an accentuation of the surface wrinkles and fine lines is observed. The skin has a rough feel. Finally, the skin shows decreased suppleness.

The main changes concerning the epidermis are a decrease in keratinocyte differentiation, resulting in a deficit in the proteins matrix of the cornified cell, an increase in metalloproteinases, which are proteases that degrade the extracellular matrix and that participate in ageing of the skin, and also a decrease in the synthesis of various glycosaminoglycans.

The object of the present invention is, precisely, to propose a solution to these various problems, and especially to propose novel compositions that can be used in cosmetics and pharmaceuticals to limit ageing of the skin, whether it is chronobiological or photo-induced ageing, and especially ageing generated by a decrease in the elasticity of the skin and/or by a degradation of collagen in the structure of the tissues and/or by a decrease in glycosaminoglycan synthesis.

U.S. Pat. No. 6,987,128 describes the efficacy in this respect of N-acylamino amide derivatives. More specifically, these derivatives prove to be especially advantageous with regard to their ability to slow down the degradation activity of the elastic fibres of the intercellular spaces.

Similarly, sugars and sugar derivatives are products that have already been exploited for various purposes for the formulation of cosmetic compositions intended either for skincare or for caring for and/or washing keratin fibres. Thus, in U.S. Pat. No. 6,495,147, D-xylose and derivatives thereof are proposed for the purposes of preparing cosmetic or pharmaceutical products aimed at improving the functionality of epidermal cells.

Certain C-glycoside derivatives have demonstrated advantageous biological properties, in particular for activating the synthesis of glycosaminoglycans and collagen VII. Such compounds are especially described in U.S. Pat. No. 7,049,300.

These compounds are more particularly represented by the formula: 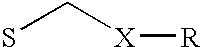
in which S represents a monosaccharide or a polysaccharide, R represents various linear or cyclic radicals and the group X may represent a group chosen from: —CO—, —CH(NR₁R₂)—, CHR′—, —C(═CHR′)—, with R′ possibly representing various radicals, including the hydroxyl radical.

The inventors have found, surprisingly, that a compound of the N-acylamino amide family, in combination with a C-glycoside derivative, has synergistic activity for stimulating keratinocyte differentiation and thus for increasing the thickness and quality of the epidermis.

Consequently, a first aspect of the invention relates to a cosmetic and/or dermatological composition combining, in a physiologically acceptable medium, at least one N-acylamino amide derivative and at least one C-glycoside derivative.

Another subject of the invention is the method, especially the cosmetic method for preventively or curatively treating the signs of ageing of bodily or facial skin, whether it is chronobiological or photo-induced ageing, and especially ageing generated by a decrease in skin elasticity and/or by a degradation of the collagen in the structure of the tissues, comprising at least a step consisting in applying to said skin a composition comprising at least one N-acylamino amide derivative in combination with at least one C-glycoside derivative.

Another subject of the invention is the method, especially the cosmetic method for preventively or curatively treating aged skin showing at least one of the following signs of aging chosen among wrinkles and/or fine lines, wizened skin, lack of skin elasticity and/or tonus, thinning of the dermis, the degradation of collagen fibres, flaccid skin, thinned skin, and internal degradations of the skin following exposure to ultraviolet radiation comprising at least a step consisting in applying to said skin a composition comprising at least one N-acylamino amide derivative in combination with at least one C-glycoside derivative.

Another subject of the invention is the method, especially the cosmetic method for inhibiting the activity of elastases and/or for limiting and/or combating the degradation of elastic fibres of skin, comprising at least a step consisting in applying to said skin a composition comprising at least one N-acylamino amide derivative in combination with at least one C-glycoside.

Another subject of the invention is the method, especially the cosmetic method for increasing keratinocyte differentiation comprising at least a step consisting in applying to said skin composition comprising at least one N-acylamino amide derivative in combination with at least one C-glycoside derivative.

It has been shown in the context of the present invention, unexpectedly, that a combination of at least one C-glycoside derivative with at least one N-acylamino amide derivative as defined hereinbelow induces an increase in TGk in the skin.

TGk is an enzyme belonging to the transpeptidase family. It is a calcium-dependent enzyme that catalyzes the formation of ε(γ-glutamyl)lysine isopeptide bridges between epidermal proteins and thus promotes the formation of horny sheaths.

The combination according to the invention thus allows an improvement in keratinocyte differentiation and in the maturation and cohesion of the horny layer.

According to another aspect, the invention relates to the cosmetic method for improving the barrier function of the skin comprising at least a step consisting in applying to said skin a composition comprising at least a combination of at least one C-glycoside derivative with at least one N-acylamino amide derivative.

Impairment in the skin barrier may take place in the presence of external attacking factors such as irritants (detergents, acids, bases, oxidizing agents, reducing agents, concentrated solvents, or toxic gases or fumes), mechanical stresses (friction, impacts, abrasion, tearing of the surface, projection of dusts or particles, shaving or hair removal), thermal or climatic imbalances (cold, dryness or radiation), xenobiotics (undesirable microorganisms, allergens) or internal attacking factors such as psychological stress.

This impairment in the skin barrier may especially be reflected by cutaneous discomfort, sensory phenomena and especially unpleasant phenomena. The sensory nerve endings of the surface of the skin or the scalp, especially the C fibres of neurons, the chemical, thermal and mechanical receptors or sensors or the pressure receptors are in fact partially “denuded”. The person may then feel a sensation of cutaneous discomfort that may especially be manifested by stinging, tautness, sensations of heating or itching.

Thus, according to yet another aspect, the invention relates to the cosmetic method for reinforcing the resistance of the skin to external attack, in particular to pollution and to pollutants (such as particles, ozone, nitrogen oxides, sulfur oxides and/or heavy metals, for instance cobalt, cadmium or mercury) or to free radicals, to sudden changes in temperature or relative humidity of the atmosphere, to the wind, to conditioned air, to mechanical stresses, especially irritation caused by certain rough fabrics or excessively aggressive hygiene comprising at least a step consisting in applying to said skin a composition comprising at least a combination of at least one C-glycoside derivative with at least one N-acylamino amide derivative. This method may also be directed towards reinforcing the resistance of the skin to internal stresses, especially to changes induced by physiological stress or fatigue that have an impact on the surface state of the skin.

The combination of at least one C-glycoside derivative with at least one N-acylamino amide derivative according to the invention will thus be particularly useful for stimulating natural moisturization and the radiance of the skin, which appears more luminous and softer and/or for improving and/or reducing the skin's microrelief.

A subject of the present invention is thus also a cosmetic treatment process for maintaining or promoting the moisturization of the skin and/or mucous membranes, for reinforcing the barrier function of the skin and/or mucous membranes, and for promoting the resistance of the skin and/or mucous membranes to external attacking factors associated with the environment or to changes caused by intrinsic stress, comprising the application of an effective amount of a combination of at least one C-glycoside derivative with at least one N-acylamino amide derivative.

The combination will be applied to the part of the skin to be treated, in particular to the face, the neck or the hands, daily or several times a day. The application will be renewed every day over a variable period according to the desired effects, generally from 3 to 6 weeks, but may be prolonged or pursued continuously.

A subject of the invention is also the cosmetic method to prevent and/or treat skin in need of an agent for promoting keratinocyte differentiation and/or comification comprising at least a step consisting in applying to said skin a composition containing a physiologically acceptable medium and comprising at least a combination of at least one C-glycoside derivative with at least one N-acylamino amide derivative; in particular, the invention is directed towards said method wherein this combination acts as an agent for maintaining and/or reinforcing the epidermis, for maintaining the integrity and/or thickness of the various layers of the epidermis, in particular of the stratum corneum, for maintaining and/or improving the barrier function of the skin, and/or for promoting the natural moisturization of the skin.

Said combination is particularly advantageous as a care agent for promoting and/or restoring cohesion of the horny layer and thus for ensuring good homogeneity of the surface of the skin and the lips, thus especially favouring a better aesthetic makeup result for makeup compositions (homogeneity and staying power).

The present invention is also directed to a pharmaceutical method for treating skin pathologies associated with an abnormally low comification of the horny layer, comprising at least a step consisting in applying to the skin a composition containing a physiologically acceptable medium and comprising at least a combination of at least one C-glycoside derivative with at least one N-acylamino amide derivative.

It is also advantageous in processes for preparing reconstructed epidermides and/or skins.

Another subject of the invention is the dermatological method for combating impairment of the barrier function and/or changes in the integrity of the stratum corneum, comprising at least a step consisting in applying to the skin a composition containing a physiologically acceptable medium and comprising at least a combination of at least one C-glycoside derivative with at least one N-acylamino amide derivative.

Another of the aspects of the invention relates to the dermatological method for preventing and/or treating the signs of ageing of the skin and/or for stimulating the synthesis of collagen, in particular of procollagen I and/or for increasing keratinocyte differentiation, comprising at least a step consisting in applying to the skin a composition containing a physiologically acceptable medium and comprising at least a combination of at least one C-glycoside derivative with at least one N-acylamino amide derivative.

Another subject of the invention is a cosmetic process for treating bodily or facial skin, including the scalp, in which a cosmetic composition as defined above is applied to the skin.

According to yet another of its aspects, the invention relates to a therapeutic treatment process for preventing and/or treating the signs of ageing of the skin, comprising the administration to an individual of at least one N-acylamino amide derivative in combination with at least one C-glycoside derivative.

Thus, the results featured in the examples below show that by combining a C-glycoside derivative with an N-acetylamino amide derivative, keratinocyte differentiation is increased. This increase strengthens the stratification of the epidermis, which has the effect of reinforcing the barrier function of the epidermis and thus of protecting the skin against environmental damage.

N-Acylamino Amide Derivative

The compounds of this type that may be used in the present invention correspond to formula (I) below:

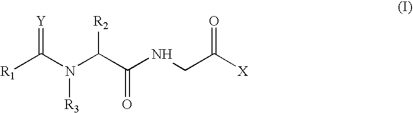

in which:
the radical Y represents O or S,
the radical R₁ represents:
(i) a hydrogen atom;
(ii) a linear, branched or cyclic, saturated or unsaturated hydrocarbon-based radical containing 1 to 18 carbon atoms,

optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; —SO₂—OR;

with R and R′ representing, independently of each other, a linear, branched or cyclic, saturated or unsaturated hydrocarbon-based radical containing 1 to 6 carbon atoms, which is optionally halogenated, or even perhalogenated,

the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
(iii) a radical chosen from the following radicals: —OR; —NH₂; —NHR; —NRR′; —NH—COR; —COOR; —COR;

with R and R′ representing, independently of each other, a linear, branched or cyclic, saturated or unsaturated hydrocarbon-based radical containing 1 to 6 carbon atoms, which is optionally halogenated, or even perhalogenated,

the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₂ represents a linear, branched or cyclic, saturated or unsaturated hydrocarbon-based radical containing 1 to 18 carbon atoms,

optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR;

with R and R′ representing, independently of each other, a linear, branched or cyclic, saturated or unsaturated hydrocarbon-based radical containing 1 to 6 carbon atoms, which is optionally halogenated, or even perhalogenated,

the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₃ represents a radical chosen from those of formula (II) or (III):
   -A-C₆H_{(5-y)}—B_{y} (II)
   —C₆H_{(5-y′)}—B_{y′} (III)

in which:
y is an integer between 0 and 5 inclusive, and y′ is an integer between 1 and 5 inclusive;
A is a linear or branched, saturated or unsaturated divalent hydrocarbon-based radical containing 1 to 18 carbon atoms,

optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;

with R and R′ representing, independently of each other, a linear, branched or cyclic, saturated or unsaturated hydrocarbon-based radical containing 1 to 6 carbon atoms, which is optionally halogenated, or even perhalogenated,

the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
B represents at least one group, which may be identical or different, chosen from —OH; —OR; —O—COR; —SH; —SR; —S—COR; —NH₂; —NHR; —NRR′; —NH—COR; -Halogen; —CN; —COOR; —COR; —NO₂; —SO₂—OR, or represents a linear or branched, saturated or unsaturated hydrocarbon-based radical containing 1 to 18 carbon atoms,

optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;

with R and R′ representing, independently of each other, a linear, branched or cyclic, saturated or unsaturated hydrocarbon-based radical containing 1 to 6 carbon atoms, which is optionally halogenated, or even perhalogenated,

the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical X represents a radical chosen from —OH, —OR₄, —NH₂, —NHR₄, —NR₄R₅, —SR₄, —COOR₄; —COR₄;

with R₄ and R₅ representing, independently of each other, a linear, cyclic or branched, saturated or unsaturated hydrocarbon-based radical containing 1 to 6 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; with R and R′ representing, independently of each other, a linear, branched or cyclic, saturated or unsaturated hydrocarbon-based radical containing 1 to 6 carbon atoms, which is optionally halogenated, or even perhalogenated, the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;

the said radicals R₄ and R₅ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated.

Also included in this definition are the salts of a mineral or organic acid of the said compounds, and also optical isomers thereof, in isolated form or as a racemic mixture.

The term “linear, cyclic or branched hydrocarbon-based radical” especially means radicals of the alkyl, aryl, aralkyl, alkylaryl, alkenyl or alkynyl type.

The group C₆H₅ present in the radical R₃ should be included as a cyclic aromatic group.

Preferably, the radical Y represents oxygen.

Preferably, the radical R₁ represents hydrogen or a linear or branched, saturated or unsaturated hydrocarbon-based radical containing 1 to 12 and especially 1, 2, 3, 4, 5 or 6 carbon atoms, which is optionally substituted.

In particular, the substituents of R₁ may be chosen from —OH, —OR and/or —P(O)—(OR)₂ with R representing a linear, branched or cyclic, saturated or unsaturated hydrocarbon-based radical containing 1 to 6 carbon atoms, which is optionally halogenated, or even perhalogenated.

Preferentially, the radical R₁ represents a methyl, ethyl, propyl or isopropyl radical, optionally substituted with a group —OH or —P(O)—(OR)₂ with R representing methyl, ethyl, propyl or isopropyl.

Preferably, the radical R₂ represents a linear, branched or cyclic, saturated or unsaturated hydrocarbon-based radical containing 1 to 12 and especially 1, 2, 3, 4, 5 or 6 carbon atoms, which is optionally substituted.

In particular, the substituents of R₂ may be chosen from —OH and —OR with R representing a linear, branched or cyclic, saturated or unsaturated hydrocarbon-based radical containing 1 to 6 carbon atoms, which is optionally halogenated, or even perhalogenated.

Preferentially, the radical R₂ represents a methyl, ethyl, propyl, isopropyl, n-butyl, tert-butyl or isobutyl radical.

Preferably, the radical R₃ represents a radical of formula —C₆H_{(5-y′)}—B_{y′} for which y′=1, 2 or 3; or a radical of formula -A-C₆H_{(5-y)}—B_{y} for which y=0, 1 or 2.

Preferably, A is a linear or branched, saturated or unsaturated divalent hydrocarbon-based radical containing 1 to 12 carbon atoms, which is optionally substituted.

The substituents of A are preferably chosen from -Hal (halogen, or even perhalogen); —CN; —COOR; —NO₂; —SO₂—OR; with R representing a linear, branched or cyclic, saturated or unsaturated hydrocarbon-based radical containing 1 to 6 carbon atoms, which is optionally halogenated or even perhalogenated.

Preferably, B represents at least one group —OR; —NHR; —CN; —COOR; —COR or represents a hydrocarbon-based radical chosen from a linear or branched, saturated or unsaturated hydrocarbon-based radical containing 1 to 12 carbon atoms, which is optionally substituted.

The substituents of B are preferably chosen from -Hal (halogen, or even perhalogen); —CN; —COOR; —NO₂; —SO₂—OR; with R representing a linear, branched or cyclic, saturated or unsaturated hydrocarbon-based radical containing 1 to 6 carbon atoms, which is optionally halogenated, or even perhalogenated.

Preferentially, the radical R₃ represents a group chosen from one of the following formulae: 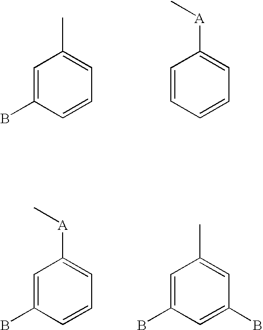
in which A and B have the above meanings.

In particular, the divalent radical A may be a methylene, an ethylene or a propylene.

Preferably, the radical B represents at least one group —OR; —NHR; —CN; —COOR; —COR for which R denotes a methyl, ethyl, propyl or isopropyl radical, or represents a hydrocarbon-based radical chosen from a methyl, ethyl, propyl or isopropyl radical, substituted with one or more halogens, especially chlorine bromine, iodine or fluorine, and preferentially totally halogenated (perhalogenated), such as perfluorinated. Mention may be made in particular of the perfluoromethyl radical (—CF₃) as being most particularly preferred.

Preferably, the radical X represents a radical chosen from —OH and —OR₄ with R₄ representing a linear, cyclic or branched, saturated or unsaturated hydrocarbon-based radical containing 1 to 6 carbon atoms, which is optionally substituted.

The substituents R₄ may be chosen from —OH and —OR with R representing a linear, branched or cyclic, saturated or unsaturated hydrocarbon-based radical containing 1 to 6 carbon atoms, which is optionally halogenated, or even perhalogenated.

Preferentially, the radical X represents a radical chosen from —OH, —OCH₃, —OC₂H₅, —O—C₃H₇ or —OC₄H₉.

Among the preferred compounds that may be used in the framework of the present invention, compounds of formula (I) may be cited, wherein
the radical Y represents oxygen,
the radical R₁ represents a methyl, ethyl, propyl or isopropyl radical, optionally substituted with a group —OH or —P(O)—(OR)₂ with R representing methyl, ethyl, propyl or isopropyl,
the radical R₂ represents a methyl, ethyl, propyl, isopropyl, n-butyl, tert-butyl or isobutyl radical,

the radical R₃ represents a group chosen from one of the following formulae:

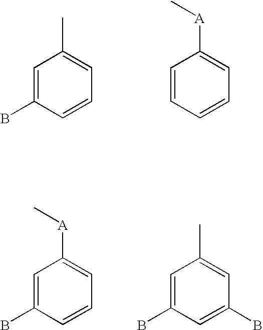

in which the divalent radical A is a methylene, an ethylene or a propylene and the radical B represents at least one group —OR; —NHR;

—CN; —COOR; —COR for which R denotes a methyl, ethyl, propyl or isopropyl radical, or represents a hydrocarbon-based radical chosen from a methyl, ethyl, propyl or isopropyl radical, substituted with one or more halogens such as perfluorinated, such as the perfluoromethyl radical (—CF₃), and
the radical X represents a radical chosen from —OH, —OCH₃, —OC₂H₅, —O—C₃H₇ or —OC₄H₉.

Among the particularly preferred compounds that may be mentioned are:
10-{2-[Acetyl(3-trifluoromethylphenyl)amino]-3-methylbutyrylamino}acetic acid,
Ethyl {2-[acetyl(3-trifluoromethylphenyl)amino]-3-methylbutyryl-amino}acetate,
[2-(Acetylbenzylamino)-3-methylbutyrylamino]acetic acid,
Ethyl [2-(acetylbenzylamino)-3-methylbutyrylamino]acetate, and
Ethyl (2-{benzyl[(diethoxyphosphoryl)acetyl]amino}-3-methylbutyryl-amino)acetate.

In particular, the compound of formula I according to the invention is a pseudodipeptide consisting of {2-[acetyl(3-trifluoromethylphenyl)amino]-3-methyl-butyrylamino}acetic acid, also known as N—[N-acetyl, N′-(3-trifluoromethyl)-phenylvalyl]glycine or N-acetyl-N-[3-(trifluoromethyl)phenyl]valyl-glycine or acetyl trifluoromethylphenylvalylglycine, or an ester thereof, especially with a C₁-C₆ alcohol.

The compounds according to the invention may be readily prepared by a person skilled in the art on the basis of his general knowledge. It is especially possible to react together a carboxylic acid, an aldehyde, an amino compound and an isonitrile, according to the Ugi reaction.

Needless to say, during the synthesis of the compounds according to the invention, and as a function of the nature of the various radicals present on the starting compounds, a person skilled in the art may take care to protect certain substituents so that they do not interfere in the sequence of reactions.

The amount of compound(s) to be used in the compositions according to the invention may be readily determined by a person skilled in the art, as a function of the nature of the compounds used, of the person to be treated and/or of the desired effect.

In general, this amount may be between 0.00001% and 20% by weight relative to the total weight of the composition, especially between 0.001% and 10% by weight, preferably between 0.05% and 5% by weight, better still between 0.1% and 2% by weight and preferentially between 0.5% and 1% by weight.

C-Glycoside Derivatives

A C-glycoside derivative that is suitable for use in the invention may be a compound of general formula (IV) below: 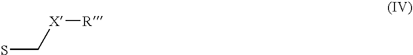
in which:

R′″ represents:
a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical;
a saturated C₁-C₂₀ and in particular C₁-C₁₀ or unsaturated C₂-C₂₀ and in particular C₂-C₁₀, or saturated or unsaturated, branched or cyclic C₃-C₂₀ and in particular C₃-C₁₀ linear hydrofluoroalkyl or perfluoroalkyl radical;
   the hydrocarbon-based chain constituting the said radicals possibly being, where appropriate, interrupted with 1, 2, 3 or more heteroatoms chosen from:
an oxygen,
a sulfur,
a nitrogen, and
a silicon,
   and possibly being optionally substituted with at least one radical chosen from:
—OR′₄,
—SR′₄,
—NR′₄R′₅,
—COOR′₄,
—CONHR′₄,
—CN,
a halogen atom,
a C₁-C₆ hydrofluoroalkyl or perfluoroalkyl radical, and/or
a C₃-C₈ cycloalkyl radical,
   with R′₄ and R′₅ possibly representing, independently of each other, a hydrogen atom or a saturated C₁-C₃₀ and in particular C₁-C₁₂ or unsaturated C₂-C₃₀ and in particular C₂-C₁₂, or a saturated or unsaturated, branched or cyclic C₃-C₃₀ and in particular C₃-C₁₂ alkyl, perfluoroalkyl or hydrofluoroalkyl radical; or a C₆-C₁₀ aryl radical,

X′ represents a radical chosen from the groups: 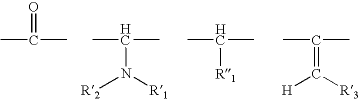
with R′₁, R′₂ and R′₃ representing, independently of each other, a hydrogen atom or a radical R′″, with R′″ as defined above, and R″₁ represents a hydrogen atom, an —OH group or a radical R′″ as defined above, R′, possibly also denoting a C₆-C₁₀ aryl radical;

S represents a monosaccharide or a polysaccharide comprising up to 20 sugar units and in particular up to 6 sugar units, in pyranose and/or furanose form and of L and/or D series, the said mono- or polysaccharide possibly being substituted with a mandatorily free hydroxyl group, and optionally one or more optionally protected amine function(s), and

the bond S—CH₂—X′ represents a bond of C-anomeric nature, which may be α or β, and also the cosmetically acceptable salts thereof, the solvates thereof such as hydrates, and the isomers thereof.

In the context of the present invention, the term “halogen” means chlorine, fluorine, bromine or iodine.

The term “aryl” denotes an aromatic ring such as phenyl, optionally substituted with one or more C₁-C₄ alkyl radicals.

The term “C₃-C₈ cycloalkyl” denotes an aliphatic ring containing from 3 to 8 carbon atoms, for example including cyclopropyl, cyclopentyl and cyclohexyl.

Among the alkyl groups that are suitable for use in the invention, mention may be made especially of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

According to one embodiment of the invention, it is possible to use a C-glycoside derivative corresponding to formula (IV) for which S may represent a monosaccharide or a polysaccharide containing up to 6 sugar units, in pyranose and/or furanose form and of L and/or D series, the said monosaccharide or polysaccharide containing at least one hydroxyl function that is mandatorily free and/or optionally one or more amine functions that are mandatorily protected, X′ and R′″ otherwise retaining all the definitions given above.

Advantageously, a monosaccharide of the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine and N-acetyl-D-galactosamine, and advantageously denotes D-glucose, D-xylose, N-acetyl-D-glucosamine or L-fucose, and in particular D-xylose.

More particularly, a polysaccharide of the invention containing up to 6 sugar units may be chosen from D-maltose, D-lactose, D-cellobiose, D-maltotriose, a disaccharide combining a uronic acid chosen from D-iduronic acid and D-glucuronic acid with a hexosamine chosen from D-galactosamine, D-glucosamine, N-acetyl-D-galactosamine and N-acetyl-D-glucosamine, an oligosaccharide containing at least one xylose advantageously chosen from xylobiose, methyl-β-xylobioside, xylotriose, xylotetraose, xylopentaose and xylohexaose and especially xylobiose, which is composed of two xylose molecules linked via a 1-4 bond.

More particularly, S may represent a monosaccharide chosen from D-glucose, D-xylose, L-fucose, D-galactose and D-maltose, especially D-xylose.

According to another embodiment of the invention, it is possible to use C-glycoside derivatives corresponding to formula (IV) for which X′ represents a group chosen from —CO—, —CH(OH)—, —CH(NR′₁R′₂)— and —CH(R′″)—, in particular —CO—, —CH(OH)—, —CH(NH₂)—, —CH(NHCH₂CH₂CH₂OH)—, —CH(NHPh)— and —CH(CH₃)—, and more particularly a —CO—, —CH(OH)— or —CH(NH₂)— group, and preferentially a —CH(OH)— group, S and R otherwise conserving all of the definitions given above.

According to another embodiment of the invention, it is possible to use a C-glycoside derivative corresponding to formula (IV) for which R′″ represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical; and optionally substituted as described above, S and X′ otherwise conserving all the definitions given above. Preferably, R′″ denotes a linear C₁-C₄ and especially C₁-C₃ radical optionally substituted with —OH, —COOH or —COOR′″₂, R′″₂ being a saturated C₁-C₄ alkyl radical, especially ethyl.

Preferentially, R′″ denotes an unsubstituted linear C₁-C₄ and especially C₁-C₂ alkyl radical, in particular ethyl.

Among the C-glycoside derivatives of formula (IV) that are preferably used are those for which:
R′″ represents a saturated C₁-C₂₀ and in particular C₁-C₁₀ or unsaturated C₂-C₂₀ and in particular C₂-C₁₀ linear alkyl radical, or a saturated or unsaturated, branched or cyclic C₃-C₂₀ and in particular C₃-C₁₀ alkyl radical, optionally substituted as described above;
S represents a monosaccharide as described above;
X′ represents —CO—, —CH(OH)—, —CH(NR′₁R′₂)— or —CH(R′″)—, as defined above.

Preferably, a C-glycoside derivative of formula (IV) is used, for which:
R′″ denotes a linear C₁-C₄ and especially C₁-C₃ radical, optionally substituted with —OH, —COOH or —COOR′″₂, R′″₂ being a saturated C₁-C₄ alkyl radical, especially ethyl;
S represents a monosaccharide as described above;
X′ represents a group chosen from —CO—, —CH(OH)—, —CH(NH₂)—, —CH(NHCH₂CH₂CH₂OH)—, —CH(NHPh)- and —CH(CH₃)—, and more particularly a —CO—, —CH(OH)— or —CH(NH₂)— group, and preferentially a —CH(OH)— group.

Preferentially, a C-glycoside derivative of formula (IV) is used, for which:
R′″ denotes an unsubstituted linear C₁-C₄ and especially C₁-C₂ alkyl radical, in particular ethyl;
S represents a monosaccharide as described above; especially D-glucose, D-xylose, N-acetyl-D-glucosamine or L-fucose, in particular D-xylose;
X′ represents a group chosen from —CO—, —CH(OH)— and —CH(NH₂)— and preferentially a CH(OH)— group.

The salts that are acceptable for the non-therapeutic use of the compounds described in the present invention comprise conventional non-toxic salts of the said compounds such as those formed from organic or inorganic acids. Examples that may be mentioned include the salts of mineral acids, such as sulfuric acid, hydrochloric acid, hydrobromic acid, hydriodic acid, phosphoric acid or boric acid. Mention may also be made of the salts of organic acids, which may comprise one or more carboxylic, sulfonic or phosphonic groups. They may be linear, branched or cyclic aliphatic acids or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

When the compound of formula (IV) comprises an acid group, neutralization of the acid group(s) may be performed with a mineral base, such as LiOH, NaOH, KOH, Ca(OH)₂, NH₄OH, Mg(OH)₂ or Zn(OH)₂; or with an organic base such as a primary, secondary or tertiary alkylamine, for example triethylamine or butylamine. This primary, secondary or tertiary alkylamine may comprise one or more nitrogen and/or oxygen atoms and may thus comprise, for example, one or more alcohol functions; mention may be made especially of amino-2-methyl-2-propanol, triethanolamine, dimethylamino-2-propanol or 2-amino-2-(hydroxymethyl)-1,3-propanediol. Mention may also be made of lysine or 3-(dimethylamino)propylamine.

The solvates that are acceptable for the compounds described in the present invention comprise conventional solvates such as those formed during the final step of preparation of the said compounds due to the presence of solvents. Examples that may be mentioned include the solvates due to the presence of water or of linear or branched alcohols, for instance ethanol or isopropanol.

Among the C-glycoside derivatives of formula (IV) used according to the invention, the ones that are most particularly considered are:
1. C-β-D-xylopyranoside-n-propan-2-one;
2. C-α-D-xylopyranoside-n-propan-2-one;
3. 1-[2-(3-hydroxypropylamino)propyl]-C-β-D-xylopyranose;
4. 1-[2-(3-hydroxypropylamino)propyl]-C-α-D-xylopyranose;
5. C-β-D-xylopyranoside-2-hydroxypropane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-phenylaminopropane;
10. C-α-D-xylopyranoside-2-phenylaminopropane;
11. ethyl 3-methyl-4-(C-β-D-xylopyranoside)butyrate;
12. ethyl 3-methyl-4-(C-α-D-xylopyranoside)butyrate;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-phenylaminohexanoic acid;
20. 6-(C-α-D-xylopyranoside)-5-phenylaminohexanoic acid;
21. 1-(C-β-D-xylopyranoside)hexane-2,6-diol;
22. 1-(C-α-D-xylopyranoside)hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-α-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-phenylaminopentanoic acid;
30. 5-(C-α-D-xylopyranoside)-4-phenylaminopentanoic acid;
31. 1-(C-β-D-xylopyranoside)pentane-2,5-diol;
32. 1-(C-α-D-xylopyranoside)pentane-2,5-diol;
33. 1-(C-β-D-fucopyranoside)propan-2-one;
34. 1-(C-α-D-fucopyranoside)propan-2-one;
35. 1-(C-β-L-fucopyranoside)propan-2-one;
36. 1-(C-α-L-fucopyranoside)propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-D-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-phenylaminopropane;
46. 1-(C-α-D-fucopyranoside)-2-phenylaminopropane;
47. 1-(C-β-L-fucopyranoside)-2-phenylaminopropane;
48. 1-(C-α-L-fucopyranoside)-2-phenylaminopropane;
49. ethyl 3-methyl-4-(C-β-D-fucopyranoside)butyrate;
50. ethyl 3-methyl-4-(C-α-D-fucopyranoside)butyrate;
51. ethyl 3-methyl-4-(C-β-L-fucopyranoside)butyrate;
52. ethyl 3-methyl-4-(C-α-L-fucopyranoside)butyrate;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-α-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C-β-L-fucopyranoside)-5-aminohexanoic acid;
64. 6-(C-α-L-fucopyranoside)-5-aminohexanoic acid;
65. 1-(C-β-D-fucopyranoside)hexane-2,6-diol;
66. 1-(C-α-D-fucopyranoside)hexane-2,6-diol;
67. 1-(C-β-L-fucopyranoside)hexane-2,6-diol;
68. 1-(C-α-L-fucopyranoside)hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside)-4-ketopentanoic acid;
72. 5-(C-α-L-fucopyranoside)-4-ketopentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid
79. 5-(C-β-L-fucopyranoside)-4-aminopentanoic acid;
80. 5-(C-α-L-fucopyranoside)-4-aminopentanoic acid;
81. 1-(C-β-D-fucopyranoside)pentane-2,5-diol;
82. 1-(C-α-D-fucopyranoside)pentane-2,5-diol;
83. 1-(C-β-L-ficopyranoside)pentane-2,5-diol;
84. 1-(C-α-L-fucopyranoside)pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-phenylaminopropane;
90. 1-(C-α-D-glucopyranosyl)-2-phenylaminopropane;
91. ethyl 3-methyl-4-(C-β-D-glucopyranosyl)butyrate;
92. ethyl 3-methyl-4-(C-α-D-glucopyranosyl)butyrate;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-phenylaminohexanoic acid;
100. 6-(C-α-D-glucopyranosyl)-5-phenylaminohexanoic acid;
101. 1-(C-β-D-glucopyranosyl)hexane-2,6-diol;
102. 1-(C-α-D-glucopyranosyl)hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-hydroxypentanoic acid;
109. 6-(C-β-D-glucopyranosyl)-5-phenylaminopentanoic acid;
110. 6-(C-α-D-glucopyranosyl)-5-phenylaminopentanoic acid;
111. 1-(C-β-D-glucopyranosyl)pentane-2,5-diol;
112. 1-(C-α-D-glucopyranosyl)pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-α-D-galactopyranosyl)-2-aminopropane;
117. 1-(C-β-D-galactopyranosyl)-2-phenylaminopropane;
118. 1-(C-α-D-galactopyranosyl)-2-phenylaminopropane;
119. ethyl 3-methyl-4-(β-D-galactopyranosyl)butyrate;
120. ethyl 3-methyl-4-(α-D-galactopyranosyl)butyrate;
121. 6-(C-β-D-galactopyranosyl)-5-ketohexanoic acid;
122. 6-(C-α-D-galactopyranosyl)-5-ketohexanoic acid;
123. 6-(C-β-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-phenylaminohexanoic acid;
128. 6-(C-α-D-galactopyranosyl)-5-phenylaminohexanoic acid;
129. 1-(C-β-D-galactopyranosyl)hexane-2,6-diol;
130. 1-(C-α-D-galactopyranosyl)hexane-2,6-diol;
131. 6-(C-β-D-galactopyranosyl)-5-ketopentanoic acid;
132. 6-(C-α-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-phenylaminopentanoic acid;
138. 6-(C-α-D-galactopyranosyl)-5-phenylaminopentanoic acid;
139. 1-(C-β-D-galactopyranosyl)pentane-2,6-diol;
140. 1-(C-α-D-galactopyranosyl)pentane-2,6-diol;
141. 1-(C-β-D-fucofuranosyl)propan-2-one;
142. 1-(C-α-D-fucofuranosyl)propan-2-one;
143. 1-(C-β-L-fucofuranosyl)propan-2-one;
144. 1-(C-α-L-fucofuranosyl)propan-2-one;
145. 3′-(acetamido-C-β-D-glucopyranosyl)propane-2′-one;
146. 3′-(acetamido-C-α-D-glucopyranosyl)propane-2′-one;
147. 1-(acetamido-C-β-D-glucopyranosyl)-2-hydroxylpropane;
148. 1-(acetamido-C-β-D-glucopyranosyl)-2-aminopropane;
149. 1-(acetamido-C-β-D-glucopyranosyl)-2-phenylaminopropane;
150. 1-(acetamido-C-α-D-glucopyranosyl)-2-phenylaminopropane;
151. ethyl 3-methyl-4-(acetamido-C-β-D-glucopyranosyl)butyrate;
152. ethyl 3-methyl-4-(acetamido-C-α-D-glucopyranosyl)butyrate;
153. 6-(acetamido-C-β-D-glucopyranosyl)-5-ketohexanoic acid;
154. 6-(acetamido-C-α-D-glucopyranosyl)-5-ketohexanoic acid;
155. 6-(acetamido-C-β-D-glucopyranosyl)-5-hydroxyhexanoic acid;
156. 6-(acetamido-C-α-D-glucopyranosyl)-5-hydroxyhexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-phenylaminohexanoic acid;
160. 6-(acetamido-C-α-D-glucopyranosyl)-5-phenylaminohexanoic acid;
161. 1-(acetamido-C-β-D-glucopyranosyl)hexane-2,6-diol;
162. 1-(acetamido-C-α-D-glucopyranosyl)hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-phenylaminopentanoic acid;
170. 6-(acetamido-C-α-D-glucopyranosyl)-5-phenylaminopentanoic acid;
171. 1-(acetamido-C-β-D-glucopyranosyl)pentane-2,5-diol;
172. 1-(acetamido-C-α-D-glucopyranosyl)pentane-2,5-diol.

As non-limiting illustrations of C-glycoside derivatives that are more particularly suitable for use in the invention, mention may be made especially of the following derivatives:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane
1-(C-β-D-fucofuranosyl)propan-2-one,
1-(C-α-D-fucofuranosyl)propan-2-one
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof and mixtures thereof.

According to one embodiment, C-β-D-xylopyranoside-2-hydroxypropane or C-α-D-xylopyranoside-2-hydroxypropane, and better still C-β-D-xylopyranoside-2-hydroxypropane, may advantageously be used for the preparation of a composition according to the invention.

According to one particular embodiment, the C-glycoside derivative is C-β-D-xylopyranoside-2-hydroxypropane in the form of a solution at 30% by weight of active material in a water/propylene glycol mixture (60%/40% by weight) such as the product sold by Chimex under the trade name Mexoryl SBB®.

Needless to say, according to the invention, a C-glycoside derivative corresponding to formula (IV) may be used alone or as a mixture with other C-glycoside derivatives in any proportion.

A C-glycoside derivative that is suitable for use in the invention may especially be obtained via the synthetic method described in document U.S. Pat. No. 7,049,300.

The amount of C-glycoside derivative to be used in a composition according to the invention depends on the desired cosmetic or therapeutic effect, and may thus vary within a wide range.

A person skilled in the art may readily determine the appropriate amounts, on the basis of his general knowledge.

A composition according to the invention may comprise a C-glycoside derivative in a proportion of about from 0.0001% to about 25% by weight of active material relative to the total weight of the composition, in particular from about 0.001% to about 10% by weight of active material, and more particularly from about 0.05% to about 5% by weight of C-glycoside derivative active material relative to the total weight of the composition.

According to one embodiment, a composition in accordance with the invention may advantageously comprise as N-acylamino amide derivative, {2-[acetyl(3-trifluoro-methylphenyl)amino]-3-methylbutyrylamino}acetic acid, or an ester thereof, for instance ethyl {2-[acetyl(3-trifluoromethylphenyl)amino]-3-methylbutyrylamino}acetate, and as C-glycoside derivative, at least C-β-D-xylopyranoside-2-hydroxypropane.

According to one variant of the invention, the N-acylamino amide derivative and the C-glycoside derivative may be combined in a weight ratio varying from 0.1 to 20; in particular from 0.5 to 10; more particularly from 0.8 to 2, for example of 1.

The two types of derivative as defined above may especially be used, in combination, in a composition that comprises a physiologically acceptable medium, especially in a cosmetic or dermatological composition that therefore moreover comprises a cosmetically or pharmaceutically acceptable medium.

The physiologically acceptable medium in which the compounds according to the invention may be used, and also its constituents, their amount, the galenical form of the composition and the method for preparing it, may be chosen by a person skilled in the art on the basis of his general knowledge and as a function of the desired type of composition.

In general, this medium may be anhydrous or aqueous. It may thus comprise an aqueous phase and/or a fatty phase.

For application to the skin, the composition may especially have the form of an aqueous or oily solution; a dispersion of the lotion or serum type; emulsions of liquid or semi-liquid consistency of the milk type obtained by dispersing a fatty phase in an aqueous phase (O/W) or conversely (W/O); suspensions or emulsions of soft consistency of the aqueous or anhydrous cream or gel type; microcapsules or microparticles; vesicular dispersions of ionic and/or nonionic type.

For application to the hair, the composition may be in the form of aqueous, alcoholic or aqueous-alcoholic solutions; in the form of creams, gels, emulsions or mousses; in the form of aerosol compositions also comprising a pressurized propellant.

When the composition is in aqueous form, especially in the form of an aqueous dispersion, emulsion or solution, it may comprise an aqueous phase, which may comprise water, a floral water and/or a spring water.

The said aqueous phase may also comprise alcohols such as C₁-C₆ monoalcohols and/or polyols such as glycerol, butylene glycol, isoprene glycol, propylene glycol or polyethylene glycol.

When the composition according to the invention is in the form of an emulsion, it may also optionally comprise a surfactant, preferably in an amount of from 0.01% to 30% by weight, relative to the total weight of the composition. The composition according to the invention may also comprise at least one coemulsifier, which may be chosen from oxoethylenated sorbitan monostearate, fatty alcohols such as stearyl alcohol or cetyl alcohol, or fatty acid esters of polyols such as glyceryl stearate.

The composition according to the invention may also comprise a fatty phase, especially consisting of fatty substances that are liquid at 25° C., such as volatile or non-volatile oils of animal, plant, mineral or synthetic origin; fatty substances that are solid at 25° C., such as waxes of animal, plant, mineral or synthetic origin; pasty fatty substances; gums; mixtures thereof.

The volatile oils are generally oils having, at 25° C., a saturating vapour pressure at least equal to 0.5 millibar (50 Pa).

Among the constituents of the fatty phase, mention may be made of:
cyclic volatile silicones containing from 3 to 8 and preferably from 4 to 6 silicon atoms,
cyclocopolymers of the dimethylsiloxane/methylalkylsiloxane type,
linear volatile silicones containing from 2 to 9 silicon atoms,
volatile hydrocarbon-based oils, such as isoparaffins and especially isododecane and fluoro oils,
poly(C₁-C₂₀)alkylsiloxanes and especially those containing trimethylsilyl end groups, among which mention may be made of linear polydimethylsiloxanes and alkylmethylpolysiloxanes such as cetyl dimethicone (CTFA name),
silicones modified with optionally fluorinated aliphatic and/or aromatic groups, or with functional groups such as hydroxyl, thiol and/or amine groups,
phenyl silicone oils,
oils of animal, plant or mineral origin, and especially animal or plant oils formed from fatty acid esters of polyols, in particular liquid triglycerides, for example sunflower oil, corn oil, soybean oil, marrow oil, grapeseed oil, sesameseed oil, hazelnut oil, apricot oil, almond oil or avocado oil; fish oils, glyceryl tricaprocaprylate, or plant or animal oils of formula R₁COOR₂ in which R₁ represents a higher fatty acid residue containing from 7 to 19 carbon atoms and R₂ represents a branched hydrocarbon-based chain containing from 3 to 20 carbon atoms, for example purcellin oil; liquid paraffin, liquid petroleum jelly, perhydrosqualene, wheatgerm oil, beauty-leaf oil, sesameseed oil, macadamia oil, grapeseed oil, rapeseed oil, coconut oil, groundnut oil, palm oil, castor oil, jojoba oil, olive oil or cereal germ oils; fatty acid esters; alcohols; acetylglycerides; octanoates, decanoates or ricinoleates of alcohols or of polyalcohols; fatty acid triglycerides; glycerides;
fluoro and perfluoro oils,
silicone gums;
waxes of animal, plant, mineral or synthetic origin, such as microcrystalline waxes, paraffin wax, petrolatum, petroleum jelly, ozokerite or montan wax; beeswax, lanolin and derivatives thereof; candelilla wax, ouricury wax, carnauba wax, Japan wax, cocoa butter, cork fibre wax or sugarcane wax; hydrogenated oils that are solid at 25° C., ozokerites, fatty esters and glycerides that are solid at 25° C.; polyethylene waxes and the waxes obtained by Fischer-Tropsch synthesis; hydrogenated oils that are solid at 25° C.; lanolins; fatty esters that are solid at 25° C.; silicone waxes; fluoro waxes.

In a known manner, the composition according to the invention may comprise adjuvants that are common in the field under consideration, such as hydrophilic or lipophilic gelling agents, hydrophilic or lipophilic additives, active agents, especially hydrophilic or lipophilic cosmetic or pharmaceutical active agents, preserving agents, antioxidants, solvents, fragrances, fillers, pigments, nacres, UV-screening agents, odour absorbers and dyes. Depending on their nature, these adjuvants may be introduced into the fatty phase, into the aqueous phase and/or into lipid spherules.

The nature and amount of these adjuvants may be chosen by a person skilled in the art on the basis of his general knowledge, so as to obtain the presentation form desired for the composition. In any case, a person skilled in the art will take care to select all the optional additional compounds and/or the amount thereof such that the advantageous properties of the composition according to the invention are not, or are not substantially, adversely affected by the envisaged addition.

The cosmetic or pharmaceutical compositions according to the invention may especially be in the form of a composition for caring for and/or treating ulcerated areas or areas that have undergone cutaneous stress or microstress, especially generated by exposure to UV and/or contact with an irritant product.

Thus, the compositions according to the invention may especially be in the form of:
a care, treatment, cleansing or protective product for facial or bodily skin, including the scalp, such as a facial or body care composition (day care, night care or moisturizing care); a facial anti-wrinkle or anti-ageing composition; a facial matting composition; a composition for irritated skin; a makeup-removing composition; a body milk, especially a moisturizing milk and optionally an after-sun milk;
an antisun composition, artificial tanning (self-tanning) composition or after-sun care composition;
a haircare composition, and especially an antisun cream or gel; a scalp care composition, especially for preventing hair loss or for restoring the hair;
a makeup product for facial skin, the body or the lips, such as a foundation, a tinted cream, a makeup rouge, an eyeshadow, a loose or compact powder, a concealer stick, a cover stick, a lipstick or a lipcare product.

The compositions according to the invention find a preferred application as compositions for facial skincare, of anti-wrinkle or anti-ageing type, and as antisun or after-sun compositions.

The composition used according to the invention may also contain other active agents, and especially at least one compound chosen from: moisturizers; depigmenting agents; anti-glycation agents; NO-synthase inhibitors; agents for stimulating the synthesis of dermal or epidermal macromolecules and/or for preventing their degradation; agents for stimulating fibroblast proliferation and/or for stimulating keratinocyte differentiation; muscle relaxants; tensioning agents; antipollution agents and/or free-radical scavengers, sunscreens and mixtures thereof.

The term “moisturizer” means:

a compound that acts on the barrier function, in order to maintain the moisturization of the stratum corneum, or an occlusive compound. Mention may be made of ceramides, sphingoid-based compounds, lecithins, glycosphingolipids, phospholipids, cholesterol and derivatives thereof, phytosterols (stigmasterol, β-sitosterol or campesterol), essential fatty acids, 1,2-diacylglycerol, 4-chromanone, pentacyclic triterpenes such as ursolic acid, liquid petroleum jelly and lanolin;

or a compound that directly increases the water content of the stratum corneum, such as threalose and derivatives thereof, hyaluronic acid and derivatives thereof, glycerol, pentanediol, sodium pidolate, serine, xylitol, sodium lactate, polyglyceryl acrylate, ectoin and derivatives thereof, chitosan, oligosaccharides and polysaccharides, cyclic carbonates, N-lauroylpyrrolidonecarboxylic acid and N-α-benzoyl-L-arginine;

or a compound that activates the sebaceous glands, such as DHEA, 7-oxide and/or 17-alkyl derivatives thereof, sapogenins and vitamin D and derivatives thereof.

The depigmenting agents that may be incorporated into the composition according to the present invention comprise, for example, the following compounds: kojic acid; ellagic acid; arbutin and derivatives thereof such as those described in U.S. Pat. No. 6,306,376 and U.S. Pat. No. 5,346,693; hydroquinone, without this list being limiting.

The term “anti-glycation agent” means a compound that can prevent and/or reduce the glycation of skin proteins, in particular of dermal proteins such as collagen.

The agents for stimulating keratinocyte differentiation comprise, for example, minerals such as calcium; the extract of lupin sold by the company Silab under the trade name Photopreventine®; the sodium beta-sitosteryl sulfate sold by the company Seporga under the trade name Phytocohesine; and the extract of corn sold by the company Solabia under the trade name Phytovityl®; and lignans such as secoisolariciresinol.

The composition according to the invention may comprise dermo-decontracting agents, among which mention may be made in particular of alverine and salts thereof, especially alverine citrate, sapogenins such as diosgenin and natural extracts containing it (such as extracts of wild yam), certain secondary and tertiary carbonyl amines, organic or mineral salts of metals, in particular manganese gluconate, adenosine, and also the hexapeptide argireline R sold by the company Lipotec. Mention may also be made of certain fragrancing compositions with a dermo-decontracting effect.

A subject of the present invention is also a cosmetic process for treating bodily or facial skin, including the scalp, in which a cosmetic composition comprising an effective amount of at least one compound of formula (I) is applied to the skin, left in contact with the skin and then optionally rinsed off.

The cosmetic treatment process of the invention may be especially performed by applying the cosmetic compositions as defined above, according to the usual technique for using these compositions. For example: application of creams, gels, sera, lotions, makeup-removing milks or antisun compositions to the skin or to dry hair; application of a scalp lotion to wet hair; application of toothpaste to the gums.

The invention is illustrated in further detail in the examples that follow, which are presented as non-limiting illustrations of the field of the invention.

The tests performed below use normal human epidermal keratinocytes (NHEK) of R3KO15 type obtained from breast surgery.

The activity of a composition in accordance with the invention is tested with regard to these keratinocytes by assaying the keratinocyte transglutamase (TGk) activity after prolonged contact of the keratinocytes with the test composition, the increase in TGk activity sanctioning a stimulation of keratinocyte differentiation.

The activity is tested in an SFM medium available from Invitrogen under the reference 17005034.

Its composition is as follows:
EGF 0.25 ng/ml,
pituitary extract (PE) 25 μg/ml (Invitrogen 3700015),
gentamycin 25 μg/ml (Sigma G1397)
optionally supplemented with 1.5 mM final calcium chloride (Prolabo 22317297).

The C-glycoside derivative used is C-β-D-xylopyranoside-2-hydroxypropane manufactured by the company Chimex under the trade name Mexoryl SBB®. It is in the form of a solution at 30% by weight of active material in a 60/40 water/propylene glycol mixture.

The N-acylamino amide derivative tested is {2-[acetyl(3-trifluoromethyl-phenyl)amino]-3-methylbutyrylamino}acetic acid. This compound is prepared according to the protocol described in Example 2 of document U.S. Pat. No. 6,987,128.

The protocol followed for the activity test is as follows:

Keratinocytes (R3KO15) are precultured in SFM1x medium in 96-well plates (one plate for the test, one for the protein assay and one for the MTT). After incubation for 24 hours at 37° C., the culture medium is removed and replaced with SFM medium, which is not supplemented (calcium-free blank) or supplemented with 1.5 mM final of calcium chloride and not containing (blank) or containing the products, mixed with the test or the references.

After incubation for 48 hours at 37° C. and 5% CO₂, the culture medium is removed and the cells are again treated and then incubated for 48 hours at 37° C.

All the experimental conditions are performed in hexaplicate.

The cell lawns are washed and then sonicated, on ice, in Tris/EDTA pH 8.0 buffer.

The membrane TGk enzyme is extracted in the presence of Triton X-100 (incubation with stirring at 4° C.) and the TGk activity is then determined by measuring the covalent addition of tritiated putrescine at 2 μCi/ml final (Amersham TRK 414; 1.04 TBq/mmol, 28 Ci/mmol) to casein (acceptor protein, 2 mg/ml final), in a Tris 0.1 M, CaCl₂ 4 mM, EDTA 0.4 mM, DTT 5 mM, pH 8.3 buffer, for 2 hours at 37° C.

The casein is precipitated by addition of 20% trichloroacetic acid (TCA) containing 1 mM putrescine and the precipitates are recovered on a Skatron filter and collector (extensive washing with 5% TCA, 0.1 mM putrescine and ethanol) and the dry filters are then counted by liquid scintillation.

The proteins of each sample are assayed using the Dc Protein Assay kit (BioRad 500-0116) and according to the protocol recommended by the manufacturer.

The cell viability was evaluated by morphological observations and performance of an MTT test.

The results obtained are given in Table 1 below, the TGk activity being relative to the amount of proteins—calcium-free medium.

As a blank test, the efficacy of retinoic acid was also tested.

TABLE 1

| Treatment | Concentration | | Activity/μg | %/blank |
|---|---|---|---|---|
| Blank (−Ca) | — | | 126 | 100 |
| Blank (+Ca) | — | | 334 | 265 |
| Retinoic acid | 10⁻⁷ | M | 111 | 88 |
| C-glycoside | 3 | mM | 210 | 167 |
| | 1 | mM | 171 | 136 |
| N-acylamino amide | 0.2 | mg/ml | 104 | 82 |
| | 0.04 | mg/ml | 114 | 91 |

| | | | |
|---|---|---|---|
| C-glycosides + | 3 mM + 0.2 mg/ml | 235 | 187 |
| N-acylamino amide | 1 mM + 0.04 mg/ml | 223 | 177 |

It is noted that:
retinoic acid at 10⁻⁷ M causes a reduction in the TGk activity relative to the blank —Ca.

However, under these experimental conditions, the C-glycoside tested at 3 mM and 1 mM significantly stimulates the TGk activity (respectively, 167% and 136% of the blank, p<0.01), whereas the N-acylamino amide tested at 0.2 mg/ml and 0.04 mg/ml had no significant effect on the TGk activity.

Surprisingly, the mixture of the two active agents at the two concentrations tested significantly increases the TGk activity present in the NHEK cultures (187% and 177% of the blank, po<0.01).

This increase is the effect of a potentialization of the TGk activity.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Cosmetic and/or dermatological composition comprising, in a physiologically acceptable medium, at least one N-acylamino amide derivative and at least one C-glycoside derivative.

2. Composition according to claim 1, wherein that the N-acylamino amide derivative corresponds to the general formula (I) below: 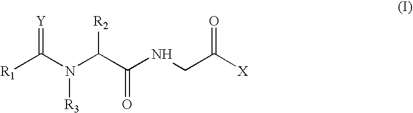
in which:
the radical Y represents O or S,
the radical R₁ represents:
(i) a hydrogen atom;
(ii) a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
(iii) a radical chosen from the following radicals: —OR; —NH₂; —NHR; —NRR′; —NH—COR; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₂ represents a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₃ represents a radical chosen from those of formula (II) or (III):
-A-C₆H_{(5-y)}—B_{y} (II)
—C₆H_{(5-y′)}—B_{y′} (III)
in which:
y is an integer between 0 and 5 inclusive, and y′ is an integer between 1 and 5 inclusive;
A is a linear or branched, saturated or unsaturated divalent hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
B represents at least one group, which may be identical or different, chosen from —OH; —OR; —O—COR; —SH; —SR; —S—COR; —NH₂; —NHR; —NRR′; —NH—COR; -Halogen; —CN; —COOR; —COR; —NO₂; —SO₂—OR, or represents a linear or branched, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical X represents a radical chosen from —OH, —OR₄, —NH₂, —NHR₄, —NR₄R₅, —SR₄, —COOR₄; —COR₄;
with R₄ and R₅ representing, independently of each other, a linear, cyclic or branched, saturated or unsaturated hydrocarbon-based radical containing 1 to 6 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; with R and R′ representing, independently of each other, a linear, branched or cyclic, saturated or unsaturated hydrocarbon-based radical containing 1 to 6 carbon atoms, which is optionally halogenated, or even perhalogenated, the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the said radicals R₄ and R₅ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated.

3. Composition according to claim 2, in which:
the radical Y represents oxygen, and/or
the radical R₁ represents hydrogen or a linear or branched, saturated or unsaturated hydrocarbon-based radical containing 1 to 12, which is optionally substituted, and/or
the substituents of R₁ are chosen from —OH, —OR and/or —P(O)—(OR)₂ with R representing a linear, branched or cyclic, saturated or unsaturated hydrocarbon-based radical containing 1 to 6 carbon atoms, which is optionally halogenated, or even perhalogenated; and/or
the radical R₂ represents a linear, branched or cyclic, saturated or unsaturated hydrocarbon-based radical containing 1 to 12, which is optionally substituted; and/or
the substituents of R₂ are chosen from —OH and —OR with R representing a linear, branched or cyclic, saturated or unsaturated hydrocarbon-based radical containing 1 to 6 carbon atoms, which is optionally halogenated, or even perhalogenated; and/or
the radical R₃ represents a radical of formula —C₆H_{(5-y′)}—B_{y′} for which y′=1, 2 or 3; or a radical of formula -A-C₆H_{(5-y)}—B_{y} for which y=0, 1 or 2; and/or
the radical A of R₃ is a linear or branched, saturated or unsaturated divalent hydrocarbon-based radical containing 1 to 12 carbon atoms, which is optionally substituted; and/or
the radical B of R₃ represents at least one group —OR; —NHR; —CN; —COOR; —COR or represents a linear or branched, saturated or unsaturated hydrocarbon-based radical containing 1 to 12 carbon atoms, which is optionally substituted; and/or
the substituents of A and/or of B are chosen from -Hal (halogen, or even perhalogen); —CN; —COOR; —NO₂; —SO₂—OR; with R representing a linear, branched or cyclic, saturated or unsaturated hydrocarbon-based radical containing 1 to 6 carbon atoms, which is optionally halogenated, or even perhalogenated; and/or
the radical X represents a radical chosen from —OH and —OR₄ with R₄ representing a linear, cyclic or branched, saturated or unsaturated hydrocarbon-based radical containing 1 to 6 carbon atoms, which is optionally substituted; and/or
the substituents of R₄ of X are chosen from —OH and —OR with R representing a linear, branched or cyclic, saturated or unsaturated hydrocarbon-based radical containing 1 to 6 carbon atoms, which is optionally halogenated, or even perhalogenated.

4. Composition according to claim 2, in which:
the radical R₁ represents a methyl, ethyl, propyl or isopropyl radical, optionally substituted with a group —OH or —P(O)—(OR)₂ with R representing methyl, ethyl, propyl or isopropyl; and/or
the radical R₂ represents a methyl, ethyl, propyl, isopropyl, n-butyl, tert-butyl or isobutyl radical; and/or
the radical R₃ represents a group chosen from one of the following formulae: 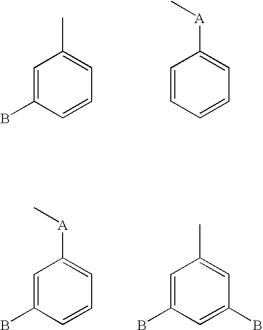
in which the divalent radical A is a methylene, an ethylene or a propylene and/or the radical B represents at least one group —OR; —NHR; —CN; —COOR; —COR for which R denotes a methyl, ethyl, propyl or isopropyl radical, or represents a hydrocarbon-based radical chosen from a methyl, ethyl, propyl or isopropyl radical substituted with one or more halogens, and/or
the radical X represents a radical chosen from —OH, —OCH₃, —OC₂H₅, —O—C₃H₇ and —OC₄H₉.

5. Composition according to claim 4, wherein R is perfluorinated.

6. Composition according to claim 1, in which the said N-acylamino amide derivative is chosen from:
{2-[Acetyl(3-trifluoromethylphenyl)amino]-3-methylbutyrylamino}acetic acid,
Ethyl {2-[acetyl(3-trifluoromethylphenyl)amino]-3-methylbutyryl-amino}acetate,
[2-(Acetylbenzylamino)-3-methylbutyrylamino]acetic acid,
Ethyl [2-(acetylbenzylamino)-3-methylbutyrylamino]acetate, and
Ethyl (2-{benzyl[(diethoxyphosphoryl)acetyl]amino}-3-methylbutyryl-amino)acetate.

7. Composition according to claim 6, in which the said N-acylamino amide derivative is {2-[acetyl(3-trifluoromethylphenyl)amino]-3-methyl-butyrylamino}acetic acid or an ester thereof.

8. Composition according to claim 1, comprising from 0.00001% to 20% by weight of N-acylamino amide derivative(s) relative to the total weight of the composition.

9. Composition according to claim 1, comprising from 0.1% to 2% by weight of N-acylamino amide derivative(s) relative to the total weight of the composition.

10. Composition according to claim 1, in which the C-glycoside derivative corresponds to the general formula (IV) below: 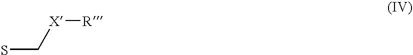
in which:
R′″ represents:
a saturated C₁-C₂₀ or unsaturated C₂-C₂₀ linear alkyl radical, or a saturated or unsaturated, branched or cyclic C₃-C₂₀ alkyl radical;
a saturated C₁-C₂₀ or unsaturated C₂-C₂₀, or saturated or unsaturated, branched or cyclic C₃-C₂₀ and linear hydrofluoroalkyl or perfluoroalkyl radical;
the hydrocarbon-based chain constituting the said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR′₄,
—SR′₄,
—NR′₄R′₅,
—COOR′₄,
—CONHR′₄,
—CN,
a halogen atom,
a C₁-C₆ hydrofluoroalkyl or perfluoroalkyl radical, and/or
a C₃-C₈ cycloalkyl radical,
with R′₄ and R′₅ possibly representing, independently of each other, a hydrogen atom or a saturated C₁-C₃₀ or unsaturated C₂-C₃₀ or a saturated or unsaturated, branched or cyclic C₃-C₃₀ alkyl, perfluoroalkyl or hydrofluoroalkyl radical; or a C₆-C₁₀ aryl radical,
X′ represents a radical chosen from the groups: 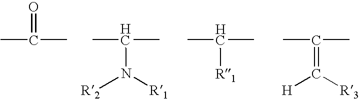
with R′₁, R′₂ and R′₃ representing, independently of each other, a hydrogen atom or a radical R′″, with R′″ as defined above, and R′₁ represents a hydrogen atom, an —OH group or a radical R′″ as defined above, R′₁ possibly also denoting a C₆-C₁₀ aryl radical;
S represents a monosaccharide or a polysaccharide comprising up to 20 sugar units 10, in pyranose and/or furanose form and of L and/or D series, the said mono- or polysaccharide possibly being substituted with a mandatorily free hydroxyl group, and optionally one or more optionally protected amine function(s), and
the bond S—CH₂—X′ represents a bond of C-anomeric nature, which may be α or β, and also the cosmetically acceptable salts thereof, the solvates thereof, and the isomers thereof.

11. Composition according to claim 10, in which S represents a monosaccharide chosen from D-glucose, D-xylose, L-fucose, D-galactose and D-maltose, especially D-xylose.

12. Composition according to claim 10, in which X′ represents a group chosen from —CO—, —CH(OH)— and —CH(NH₂)—.

13. Composition according to claim 10, in which R′″ denotes a linear C₁-C₄, optionally substituted with —OH, —COOH or —COOR′″₂, R′″₂ being a saturated C₁-C₄ alkyl radical.

14. Composition according to claim 1, in which the C-glycoside derivative is chosen from:
C-β-D-xylopyranoside-n-propan-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxylpropane,
1-(C-α-D-glucopyranosyl)-2-hydroxylpropane,
1-(C-β-D-galactopyranosyl)-2-hydroxylpropane,
1-(C-α-D-galactopyranosyl)-2-hydroxylpropane
-1-(C-β-D-fucofuranosyl)propan-2-one,
1-(C-α-D-fucofuranosyl)propan-2-one
1-(C-β-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane, isomers thereof and mixtures thereof.

15. Composition according to claim 1, in which the C-glycoside derivative is chosen from C-β-D-xylopyranoside-2-hydroxypropane and C-α-D-xylopyranoside-2-hydroxypropane.

16. Composition according to claim 1, combining C-β-D-xylopyranoside-2-hydroxypropane with {2-[acetyl(3-trifluoromethylphenyl)amino]-3-methylbutyrylamino}acetic acid or an ester thereof.

17. Composition according to claim 1, comprising the said N-acylamino amide derivative and the said C-glycoside derivative in a weight ratio varying from 0.1 to 20.

18. Composition according to claim 1, which is in the form of:
a care, treatment, cleansing or protective product for facial or bodily skin, including the scalp, such as a facial or body care composition (day care, night care or moisturizing care); a facial anti-wrinkle or anti-ageing composition; a facial matting composition; a composition for irritated skin;
an antisun composition, artificial tanning (self-tanning) composition or after-sun care composition;
a haircare composition;
a makeup product for facial skin, the body or the lips, such as a foundation, a tinted cream, a makeup rouge, an eyeshadow, a loose or compact powder, a concealer stick, a cover stick, a lipstick or a lipcare product.

19. Composition according to claim 1, which is in the form of a care composition for facial skin, of anti-wrinkle or anti-ageing type, or an antisun or after-sun composition.

20. Cosmetic method for improving the barrier function of the skin, comprising at least a step consisting in applying to said skin a composition comprising at least a combination of at least one C-glycoside derivative with at least one N-acylamino amide derivative.

21. Cosmetic method according to claim 20, wherein the N-acylamino amide derivative is as defined in claim 2 and the C-glycoside derivative is as defined in claim 10.

22. Cosmetic method for reinforcing the resistance of the skin to external attack, to free radicals, to sudden variations in atmospheric temperature or relative humidity, to the wind, to conditioned air, to mechanical stresses and to internal stress, comprising at least a step consisting in applying to said skin a composition comprising at least a combination of at least one C-glycoside derivative with at least one N-acylamino amide derivative.

23. Cosmetic method according to claim 22, wherein the N-acylamino amide derivative corresponds to the general formula (I) below: 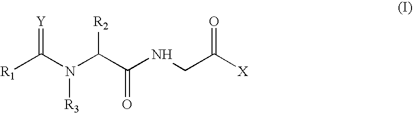
in which:
the radical Y represents O or S,
the radical R₁ represents:
(i) a hydrogen atom;
(ii) a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
(iii) a radical chosen from the following radicals: —OR; —NH₂; —NHR; —NRR′; —NH—COR; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₂ represents a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₃ represents a radical chosen from those of formula (II) or (III):
-A-C₆H_{(5-y)}—B_{y} (II)
—C₆H_{(5-y′)}—B_{y′} (III)
in which:
y is an integer between 0 and 5 inclusive, and y′ is an integer between 1 and 5 inclusive;
A is a linear or branched, saturated or unsaturated divalent hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
B represents at least one group, which may be identical or different, chosen from —OH; —OR; —O—COR; —SH; —SR; —S—COR; —NH₂; —NHR; —NRR′; —NH—COR; -Halogen; —CN; —COOR; —COR; —NO₂; —SO₂—OR, or represents a linear or branched, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical X represents a radical chosen from —OH, —OR₄, —NH₂, —NHR₄, —NR₄R₅, —SR₄, —COOR₄; —COR₄;
with R₄ and R₅ representing, independently of each other, a linear, cyclic or branched, saturated or unsaturated hydrocarbon-based radical containing 1 to 6 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; with R and R′ representing, independently of each other, a linear, branched or cyclic, saturated or unsaturated hydrocarbon-based radical containing 1 to 6 carbon atoms, which is optionally halogenated, or even perhalogenated, the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the said radicals R₄ and R₅ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated
and the C-glycoside derivative corresponds to the general formula (IV) below: 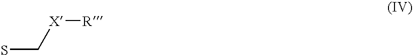
in which:
R′″ represents:
a saturated C₁-C₂₀ or unsaturated C₂-C₂₀ linear alkyl radical, or a saturated or unsaturated, branched or cyclic C₃-C₂₀ alkyl radical;
a saturated C₁-C₂₀ or unsaturated C₂-C₂₀ or saturated or unsaturated, branched or cyclic C₃-C₂₀ and linear hydrofluoroalkyl or perfluoroalkyl radical;
the hydrocarbon-based chain constituting the said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR′₄,
—SR′₄,
—NR′₄R′₅,
—COOR′₄,
—CONHR′₄,
—CN,
a halogen atom,
a C₁-C₆ hydrofluoroalkyl or perfluoroalkyl radical, and/or
a C₃-C₈ cycloalkyl radical,
with R′₄ and R′₅ possibly representing, independently of each other, a hydrogen atom or a saturated C₁-C₃₀ or unsaturated C₂-C₃₀ or a saturated or unsaturated, branched or cyclic C₃-C₃₀ alkyl, perfluoroalkyl or hydrofluoroalkyl radical; or a C₆-C₁₀ aryl radical,
X′ represents a radical chosen from the groups: 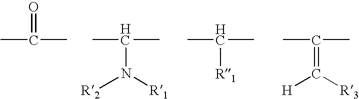
with R′₁, R′₂ and R′₃ representing, independently of each other, a hydrogen atom or a radical R′″, with R′″ as defined above, and R″, represents a hydrogen atom, an —OH group or a radical R′″ as defined above, R′, possibly also denoting a C₆-C₁₀ aryl radical;
S represents a monosaccharide or a polysaccharide comprising up to 20 sugar units and up to 6 sugar units, in pyranose and/or furanose form and of L and/or D series, the said mono- or polysaccharide possibly being substituted with a mandatorily free hydroxyl group, and optionally one or more optionally protected amine function(s), and
the bond S—CH₂—X′ represents a bond of C-anomeric nature, which may be a or
and also the cosmetically acceptable salts thereof, the solvates thereof hydrates, and the isomers thereof.

24. Cosmetic method for stimulating the natural moisturization and the radiance of the skin, and/or for improving and/or reducing the skin's microrelief, comprising at least a step consisting in applying to said skin a composition comprising at least a combination of at least one C-glycoside derivative with at least one N-acylamino amide derivative.

25. Cosmetic method according to claim 24, wherein the N-acylamino amide derivative corresponds to the general formula (I) below: 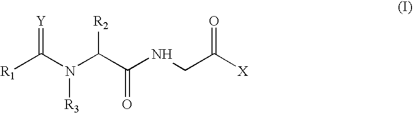
in which:
the radical Y represents O or S,
the radical R₁ represents:
(i) a hydrogen atom;
(ii) a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
(iii) a radical chosen from the following radicals: —OR; —NH₂; —NHR; —NRR′; —NH—COR; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₂ represents a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₃ represents a radical chosen from those of formula (II) or (III):
-A-C₆H_{(5-y)}—B_{y} (II)
—C₆H_{(5-y′)}—B_{y′} (III)
in which:
y is an integer between 0 and 5 inclusive, and y′ is an integer between 1 and 5 inclusive;
A is a linear or branched, saturated or unsaturated divalent hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
B represents at least one group, which may be identical or different, chosen from —OH; —OR; —O—COR; —SH; —SR; —S—COR; —NH₂; —NHR; —NRR′; —NH—COR; -Halogen; —CN; —COOR; —COR; —NO₂; —SO₂—OR, or represents a linear or branched, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical X represents a radical chosen from —OH, —OR₄, —NH₂, —NHR₄, —NR₄R₅, —SR₄, —COOR₄; —COR₄;
with R₄ and R₅ representing, independently of each other, a linear, cyclic or branched, saturated or unsaturated hydrocarbon-based radical containing 1 to 6 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; with R and R′ representing, independently of each other, a linear, branched or cyclic, saturated or unsaturated hydrocarbon-based radical containing 1 to 6 carbon atoms, which is optionally halogenated, or even perhalogenated, the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the said radicals R₄ and R₅ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated
and the C-glycoside derivative corresponds to the general formula (IV) below: 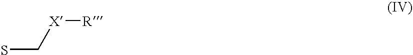
in which:
R′″ represents:
a saturated C₁-C₂₀ or unsaturated C₂-C₂₀ linear alkyl radical, or a saturated or unsaturated, branched or cyclic C₃-C₂₀ alkyl radical;
a saturated C₁-C₂₀ or unsaturated C₂-C₂₀ or saturated or unsaturated, branched or cyclic C₃-C₂₀ and linear hydrofluoroalkyl or perfluoroalkyl radical;
the hydrocarbon-based chain constituting the said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR′₄,
—SR′₄,
—NR′₄R′₅,
—COOR′₄,
—CONHR′₄,
—CN,
a halogen atom,
a C₁-C₆ hydrofluoroalkyl or perfluoroalkyl radical, and/or
a C₃-C₈ cycloalkyl radical,
with R′₄ and R′₅ possibly representing, independently of each other, a hydrogen atom or a saturated C₁-C₃₀ or unsaturated C₂-C₃₀ or a saturated or unsaturated, branched or cyclic C₃-C₃₀ alkyl, perfluoroalkyl or hydrofluoroalkyl radical; or a C₆-C₁₀ aryl radical,
X′ represents a radical chosen from the groups: 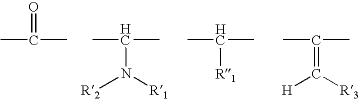
with R′₁, R′₂ and R′₃ representing, independently of each other, a hydrogen atom or a radical R′″, with R′″ as defined above, and R′₁ represents a hydrogen atom, an —OH group or a radical R′″ as defined above, R′₁ possibly also denoting a C₆-C₁₀ aryl radical;
S represents a monosaccharide or a polysaccharide comprising up to 20 sugar units and up to 6 sugar units, in pyranose and/or furanose form and of L and/or D series, the said mono- or polysaccharide possibly being substituted with a mandatorily free hydroxyl group, and optionally one or more optionally protected amine function(s), and
the bond S—CH₂—X′ represents a bond of C-anomeric nature, which may be α or β,
and also the cosmetically acceptable salts thereof, the solvates thereof hydrates, and the isomers thereof.

26. Cosmetic method for preventing and/or treating a skin in need of an agent for promoting keratinocyte differentiation and/or comification for maintaining and/or reinforcing the epidermis, for maintaining the integrity and/or the thickness of the various layers of the epidermis, for maintaining and/or improving the barrier function of the skin, and/or for promoting the natural moisturization of the skin, comprising at least a step consisting in applying to said skin a composition containing a physiologically acceptable medium and comprising at least a combination of at least one C-glycoside derivative with at least one N-acylamino amide derivative.

27. Cosmetic method according to claim 26, wherein the N-acylamino amide derivative corresponds to the general formula (I) below: 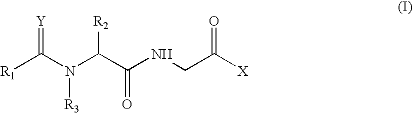
in which:
the radical Y represents O or S,
the radical R₁ represents:
(i) a hydrogen atom;
(ii) a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
(iii) a radical chosen from the following radicals: —OR; —NH₂; —NHR; —NRR′; —NH—COR; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₂ represents a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₃ represents a radical chosen from those of formula (II) or (III):
-A-C₆H_{(5-y)}—B_{y} (II)
—C₆H_{(5-y′)}—B_{y′} (III)
in which:
y is an integer between 0 and 5 inclusive, and y′ is an integer between 1 and 5 inclusive;
A is a linear or branched, saturated or unsaturated divalent hydrocarbon-based radical containing 1 to 18 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
B represents at least one group, which may be identical or different, chosen from —OH; —OR; —O—COR; —SH; —SR; —S—COR; —NH₂; —NHR; —NRR′; —NH—COR; -Halogen; —CN; —COOR; —COR; —NO₂; —SO₂—OR, or represents a linear or branched, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical X represents a radical chosen from —OH, —OR₄, —NH₂, —NHR₄, —NR₄R₅, —SR₄, —COOR₄; —COR₄;
with R₄ and R₅ representing, independently of each other, a linear, cyclic or branched, saturated or unsaturated hydrocarbon-based radical containing 1 to 6 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; with R and R′ representing, independently of each other, a linear, branched or cyclic, saturated or unsaturated hydrocarbon-based radical containing 1 to 6 carbon atoms, which is optionally halogenated, or even perhalogenated, the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the said radicals R₄ and R₅ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated and the C-glycoside derivative corresponds to the general formula (IV) below: 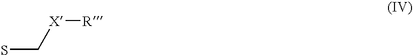
in which:
R′″ represents:
a saturated C₁-C₂₀ or unsaturated C₂-C₂₀ linear alkyl radical, or a saturated or unsaturated, branched or cyclic C₃-C₂₀ alkyl radical;
a saturated C₁-C₂₀ or unsaturated C₂-C₂₀ or saturated or unsaturated, branched or cyclic C₃-C₂₀ and linear hydrofluoroalkyl or perfluoroalkyl radical;
the hydrocarbon-based chain constituting the said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR′₄,
—SR′₄,
—NR′₄R′₅,
—COOR′₄,
—CONHR′₄,
—CN,
a halogen atom,
a C₁-C₆ hydrofluoroalkyl or perfluoroalkyl radical, and/or
a C₃-C₈ cycloalkyl radical,
with R′₄ and R′₅ possibly representing, independently of each other, a hydrogen atom or a saturated C₁-C₃₀ or unsaturated C₂-C₃₀ or a saturated or unsaturated, branched or cyclic C₃-C₃₀ alkyl, perfluoroalkyl or hydrofluoroalkyl radical; or a C₆-C₁₀ aryl radical,
X′ represents a radical chosen from the groups: 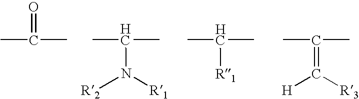
with R′₁, R′₂ and R′₃ representing, independently of each other, a hydrogen atom or a radical R′″, with R′″ as defined above, and R″₁ represents a hydrogen atom, an —OH group or a radical R′″ as defined above, R′, possibly also denoting a C₆-C₁₀ aryl radical;
S represents a monosaccharide or a polysaccharide comprising up to 20 sugar units and up to 6 sugar units, in pyranose and/or furanose form and of L and/or D series, the said mono- or polysaccharide possibly being substituted with a mandatorily free hydroxyl group, and optionally one or more optionally protected amine function(s), and
the bond S—CH₂—X′ represents a bond of C-anomeric nature, which may be a or
and also the cosmetically acceptable salts thereof, the solvates thereof hydrates, and the isomers thereof.

28. Cosmetic method for preventively or curatively treating the signs of ageing of facial or bodily skin, comprising at least a step consisting in applying to said skin a composition comprising at least a combination of at least one C-glycoside derivative with at least one N-acylamino amide derivative.

29. Cosmetic method according to claim 28, wherein the signs of ageing are chronobiological or photo-induced.

30. Cosmetic method according to claim 29, wherein ageing is generated by a decrease in skin elasticity and/or by a degradation of collagen in the structure of the tissues.

31. Cosmetic method according to claim 28, wherein the N-acylamino amide derivative corresponds to the general formula (I) below: 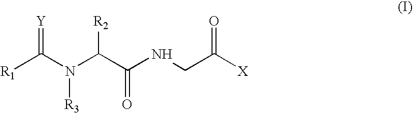
in which:
the radical Y represents O or S,
the radical R₁ represents:
(i) a hydrogen atom;
(ii) a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
(iii) a radical chosen from the following radicals: —OR; —NH₂; —NHR; —NRR′; —NH—COR; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₂ represents a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₃ represents a radical chosen from those of formula (II) or (III):
-A-C₆H_{(5-y)}—B_{y} (II)
—C₆H_{(5-y′)}—B_{y′} (III)
in which:
y is an integer between 0 and 5 inclusive, and y′ is an integer between 1 and 5 inclusive;
A is a linear or branched, saturated or unsaturated divalent hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated, the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
B represents at least one group, which may be identical or different, chosen from —OH; —OR; —O—COR; —SH; —SR; —S—COR; —NH₂; —NHR; —NRR′; —NH—COR; -Halogen; —CN; —COOR; —COR; —NO₂; —SO₂—OR, or represents a linear or branched, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical X represents a radical chosen from —OH, —OR₄, —NH₂, —NHR₄, —NR₄R₅, —SR₄, —COOR₄; —COR₄;
with R₄ and R₅ representing, independently of each other, a linear, cyclic or branched, saturated or unsaturated hydrocarbon-based radical containing 1 to 6 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; with R and R′ representing, independently of each other, a linear, branched or cyclic, saturated or unsaturated hydrocarbon-based radical containing 1 to 6 carbon atoms, which is optionally halogenated, or even perhalogenated, the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the said radicals R₄ and R₅ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated
and the C-glycoside derivative corresponds to the general formula (IV) below: 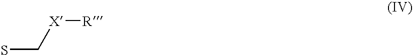
in which:
R′″ represents:
a saturated C₁-C₂₀ or unsaturated C₂-C₂₀ linear alkyl radical, or a saturated or unsaturated, branched or cyclic C₃-C₂₀ alkyl radical;
a saturated C₁-C₂₀ or unsaturated C₂-C₂₀ or saturated or unsaturated, branched or cyclic C₃-C₂₀ and linear hydrofluoroalkyl or perfluoroalkyl radical;
the hydrocarbon-based chain constituting the said radicals possibly being, where appropriate, interrupted with 1, 2, 3 or more heteroatoms chosen from:
an oxygen,
a sulfur,
a nitrogen, and
a silicon,
and possibly being optionally substituted with at least one radical chosen from:
—OR′₄,
—SR′₄,
—NR′₄R′₅,
—COOR′₄,
—CONHR′₄,
—CN,
a halogen atom,
a C₁-C₆ hydrofluoroalkyl or perfluoroalkyl radical, and/or
a C₃-C₈ cycloalkyl radical,
with R′₄ and R′₅ possibly representing, independently of each other, a hydrogen atom or a saturated C₁-C₃₀ or unsaturated C₂-C₃₀ or a saturated or unsaturated, branched or cyclic C₃-C₃₀ alkyl, perfluoroalkyl or hydrofluoroalkyl radical; or a C₆-C₁₀ aryl radical,
X′ represents a radical chosen from the groups: 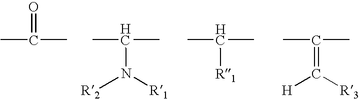
with R′₁, R′₂ and R′₃ representing, independently of each other, a hydrogen atom or a radical R′″, with R′″ as defined above, and R″, represents a hydrogen atom, an —OH group or a radical R′″ as defined above, R′, possibly also denoting a C₆-C₁₀ aryl radical;
S represents a monosaccharide or a polysaccharide comprising up to 20 sugar units and up to 6 sugar units, in pyranose and/or furanose form and of L and/or D series, the said mono- or polysaccharide possibly being substituted with a mandatorily free hydroxyl group, and optionally one or more optionally protected amine function(s), and
the bond S—CH₂—X′ represents a bond of C-anomeric nature, which may be α or β,
and also the cosmetically acceptable salts thereof, the solvates thereof hydrates, and the isomers thereof.

32. Cosmetic method for preventively or curatively treating an aged skin showing at least one of the following signs of aging chosen among wrinkles and/or fine lines, wizened skin, lack of skin elasticity and/or tonus, thinning of the dermis, the degradation of collagen fibres, flaccid skin or thinned skin and internal degradations of the skin following exposure to ultraviolet radiation, comprising at least a step consisting in applying to said skin a composition comprising at least a combination of at least one C-glycoside derivative with at least one N-acylamino amide derivative.

33. Cosmetic method for inhibiting the activity of elastases and/or for limiting and/or combating the degradation of elastic fibres, comprising at least a step consisting in applying to said skin a composition comprising at least a combination of at least one C-glycoside derivative with at least one N-acylamino amide derivative.

34. Cosmetic method for increasing keratinocyte differentiation in skin, comprising at least a step consisting in applying to said skin a composition comprising at least a combination of at least one C-glycoside derivative with at least one N-acylamino amide derivative.

35. Dermatological method for preventing and/or treating the signs of ageing of the skin and/or for stimulating the synthesis of collagen, in particular of procollagen I and/or for increasing keratinocyte differentiation, comprising at least a step consisting in applying to the skin a composition containing a physiologically acceptable medium and comprising at least a combination of at least one C-glycoside derivative as defined in claim 10, wherein the N-acylamino amide derivative corresponds to the general formula (I) below: 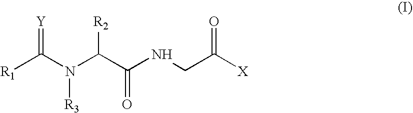
in which:
the radical Y represents O or S,
the radical R₁ represents:
(i) a hydrogen atom;
(ii) a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
(iii) a radical chosen from the following radicals: —OR; —NH₂; —NHR; —NRR′; —NH—COR; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₂ represents a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₃ represents a radical chosen from those of formula (II) or (III):
-A-C₆H_{(5-y)}—B_{y} (II)
—C₆H_{(5-y′)}—B_{y′} (III)
in which:
y is an integer between 0 and 5 inclusive, and y′ is an integer between 1 and 5 inclusive;
A is a linear or branched, saturated or unsaturated divalent hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
B represents at least one group, which may be identical or different, chosen from —OH; —OR; —O—COR; —SH; —SR; —S—COR; —NH₂; —NHR; —NRR′; —NH—COR; -Halogen; —CN; —COOR; —COR; —NO₂; —SO₂—OR, or represents a linear or branched, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical X represents a radical chosen from —OH, —OR₄, —NH₂, —NHR₄, —NR₄R₅, —SR₄, —COOR₄; —COR₄;
with R₄ and R₅ representing, independently of each other, a linear, cyclic or branched, saturated or unsaturated hydrocarbon-based radical containing 1 to 6 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; with R and R′ representing, independently of each other, a linear, branched or cyclic, saturated or unsaturated hydrocarbon-based radical containing 1 to 6 carbon atoms, which is optionally halogenated, or even perhalogenated, the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the said radicals R₄ and R₅ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated.

36. Dermatological method for combating impairment of the barrier function and/or changes in the integrity of the stratum corneum, comprising at least a step consisting in applying to the skin a composition containing a physiologically acceptable medium and comprising at least a combination of at least one C-glycoside derivative as defined in claim 10, wherein the N-acylamino amide derivative corresponds to the general formula (I) below: 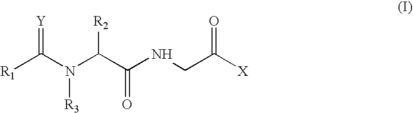
in which:
the radical Y represents O or S,
the radical R₁ represents:
(i) a hydrogen atom;
(ii) a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
(iii) a radical chosen from the following radicals: —OR; —NH₂; —NHR; —NRR′; —NH—COR; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₂ represents a linear, branched or cyclic, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₃ represents a radical chosen from those of formula (II) or (III):
-A-C₆H_{(5-y)}—B_{y} (II)
—C₆H_{(5-y′)}—B_{y′} (III)
in which:
y is an integer between 0 and 5 inclusive, and y′ is an integer between 1 and 5 inclusive;
A is a linear or branched, saturated or unsaturated divalent hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
B represents at least one group, which may be identical or different, chosen from —OH; —OR; —O—COR; —SH; —SR; —S—COR; —NH₂; —NHR; —NRR′; —NH—COR; -Halogen; —CN; —COOR; —COR; —NO₂; —SO₂—OR, or represents a linear or branched, saturated or unsaturated hydrocarbon-based radical containing 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical X represents a radical chosen from —OH, —OR₄, —NH₂, —NHR₄, —NR₄R₅, —SR₄, —COOR₄; —COR₄;
with R₄ and R₅ representing, independently of each other, a linear, cyclic or branched, saturated or unsaturated hydrocarbon-based radical containing 1 to 6 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; with R and R′ representing, independently of each other, a linear, branched or cyclic, saturated or unsaturated hydrocarbon-based radical containing 1 to 6 carbon atoms, which is optionally halogenated, or even perhalogenated, the said radicals R and R′ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the said radicals R₄ and R₅ possibly forming, together with N, a 5- or 6-membered carbon-based ring also possibly comprising at least one heteroatom chosen from O, N and/or S in the ring, and/or possibly substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated

37. Cosmetic process for treating bodily or facial skin, including the scalp, in which a cosmetic composition as defined in claim 1 is applied to the skin.

38. Cosmetic treatment process for maintaining or promoting the moisturization of the skin and/or mucous membranes, for reinforcing the barrier function of the skin and/or mucous membranes, and for promoting the resistance of the skin and/or mucous membranes to external attack associated with the environment or with changes caused by intrinsic stress, comprising the application of a composition as defined in claim 1.
